(19) United States
(12) Patent Application Publication
(10) Pub. No.: US 2016/0113377 A1
(43) Pub. Date: Apr. 28, 2016

(54) Device for Containing, Dispensing and Applying to a Substrate Content in Liquid, Gel, Cream or Paste Form

(71) Applicant: SOCOPLAN, Saint Jean de Thouars (FR)

(72) Inventors: Philippe Moussion, Misse (FR); Carl Allain, Le Perreux sur Marne (FR)

(21) Appl. No.: 14/647,790

(22) PCT Filed: Nov. 27, 2013
(86) PCT No.: PCT/FR13/52876

§ 371 Date: May 27, 2015

(30) Foreign Application Priority Data
Nov. 27, 2012 (FR) 1261318

(51) Int. Cl. A45D 34/04; B05C 17/005; A61M 35/00

(52) U.S. Cl. CPC A45D 34/042; A61M 35/003; B05C 17/00583

(57) ABSTRACT

A device for containing, dispensing and applying to a substrate content in liquid, gel, cream or paste form has a shaped semi-rigid wall, closing wall and applicator with attachment portion and application portion as extensions of one another. The shaped semi-rigid wall comprises a portion for receiving content, an attachment portion with inner projection and protection portion engaging with the attachment portion and the application portion of the applicator; the closing wall covers the semi-rigid wall; a breakable element is placed between the attachment portion and the protection portion; a content-transfer passage communicates with the content vessel and the application portion of the applicator, and is made with a structure having a radial spacing that ensures no peripheral tightness between the outer peripheral surface of the attachment portion of the applicator and the inner peripheral surface opposite the attachment portion of the shaped semi-rigid wall.

The invention relates to a device for containing, dispensing, and applying to an application surface a content in liquid, gel, cream, or paste form, this device being in the closed/associated state before if can be placed in the open/separated stats for use, and where it is full or empty of content, depending on the situation.

The field of the invention concerns single-use devices, containing a dose of content in an amount that may be suitable for single use, in particular an amount of between 0.5 ml and 10 ml, which in the open/separated state have a usage portion and a disposable portion that cannot be placed back on the usage portion.

The field of the invention also concerns an application of content that is precise in size, shape, and limits, and is small in length, surface area, and amount, the content being in liquid, gel, cream, or paste form, and suitable for being applied as though with a brush. Such content is, for example, a cosmetic product, a personal care product, a personal hygiene product, a medication, a cleaning product, a decorative product, a writing product, a masking product, a paint, an ink, a lacquer, or a varnish.

The field of the invention also concerns portable and compact devices, lightweight and small in size—for example a few centimeters for the largest dimension—which can be stored in a pocket, bag, trinket dish, or drawer.

The invention is suitable for a device requiring a secondary or supplemental container for secondary or supplemental content and for a device that can be arranged with one or mere other similar devices to form a sheet of devices.

Document WO 2010/133778 discloses non-resealable packaging comprising a rigid shell that can be deformed by pressure applied by a user, forming a storage space; a breakage area for unblocking a discharge opening; and a gripping area comprising a content escape path which is closed when the user is not applying pressure, such that the content is prevented from escaping after the packaging is opened.

Document EP 0150751 discloses a disposable dispenser comprising a container body having lateral flat, fin-like parts and a breakable neck to form a content discharge opening.

Such types of packaging only comprise a simple content delivery opening, having no true means for dispensing and application of the content and not allowing content application that is precise in size, shape, and limits, and that is small in dimensions, surface area, and amount, such as nail polish for example.

Document WO 2003/025897 discloses a device comprising a reservoir defined by a joining area connecting two groups and which comprises a dispensing orifice, and an applicator comprising a gripping member and a distribution member composed of a rod passing through the dispensing orifice, its first end being equipped with a content distribution nozzle and its second end being included in the gripping member. One of the groups is a shell comprising a cavity having a ring which the joining area is part of and one of the groups has a ring comprising a gutter. The dispensing orifice is in communication with the reservoir via a neck formed by the space between the two groups after joining, the guiter forming at feast part of the neck wall. The neck comprises a breakable structure, so that breaking a reduced joining area opens the dispensing orifice. The neck has a relatively narrow circular structure, forming a wiping member For the applicator. The dispensing orifice is closed off by a stopper element formed by joining an expansion of the ring. The gripping member of the applicator is formed by the stopper element. Such packaging is overly complex and inappropriate when there is no need to provide a wiper member.

Document FR 2837174 discloses packaging comprising: a receiving and expulsion means including a cavity provided with an outlet defined by two walls, at least one deformable. associated in a fixed and sealed manner in a peripheral region, capable of containing content of which a certain amount can be expelled through the outlet when open, if external pressure is exerted on the deformable wall; an operating means responsive to such pressure, capable of deforming the deformable wall, in the form of an additional separate part constituting a shell which is also deformable; a dispensing and application means, including a portion abutting and in communication with the outlet and an application pad; a breakable element of closing/opening the outlet, suitable for closing the outlet before first use and, after it is broken, opening the outlet for first use, consisting either of a piercing punch or a weak seam; and a protection means in the form of a removable cover mounted on the shell. Such packaging is wholly unsuitable for the field of the invention as described above.

The state of the art is also illustrated by documents US 2010/001881 and EP 1972358, which describe devices which, as above, are wholly unsuited for the field of the invention

Document DE 3122237 discloses a device comprising, with an axis, a shaped semi-rigid wall, a closing wall, and an applicator having an attachment portion and an application portion as extensions of one another, such that:
a the shaped semi-rigid wall comprises a flat peripheral edge and a recess located on one side of the edge plane and surrounded by it, having, as extensions of one another and in communication with one another, a content-receiving portion deformable by external pressure, and an attachment part with internal projection and a protection portion respectively engaging with the attachment portion and the application portion of the applicator,
the closing wall covers the shaped semi-rigid wall, is rigidly secured to the edge, and seals the recess to provide a content storage space, an attachment cavity for the attachment portion of the applicator, and a protective enclosure for the application portion of the applicator which is adapted during the opening/separation operation, to separate from said application portion in order to expose it,
a breakable element of the shaped semi-rigid wall and of the closing wall is transversely located between the attachment part and the receiving portion, and is adapted to break for the opening/separation operation,
a content-transfer passage extends along the axis, between and in communication with the content reservoir and the application portion or the applicator, so that, when using the device in the open/separated state, content is transferred from the content reservoir to the application portion of the applicator when external pressure is exerted on the receiving portion of the shaped semi-rigid wall.

The outer surface of the attachment portion of the applicator and the facing inner surface of the attachment part of the shaped semi-rigid wall are in sealing contact and act to immobilize the applicator. This seal is reinforced by the projection provided on the inner surface of the attachment part, which grips when inserted into the attachment portion of the applicator. Given this seal, the content-transfer passage is a channel traversing the attachment portion of the applicator from end to end. Thus, the content traverses the attachment portion of the applicator from end to end, from the content reservoir to the application portion of the applicator which it reaches from the inside. This arrangement, inherent in the structure of the device, does not allow efficiently applying on the application surface, as though with a brush, a content in liquid, gel, cream, or paste form, such as for example a cosmetic product, a personal care product, a personal hygiene product, a medication, a cleaning product, a decorative product, a writing product, a masking product, a paint, an ink, a lacquer, a varnish, in a precise manner concerning size, shape, and limits, and that is small in size, surface area, and amount

Document DE 10 2009 029 concerns a different device but which has a similar structure with peripheral sealing.

The first problem solved by the invention is therefore to eliminate a structure with peripheral sealing and a passage through the applicator.

In addition, and in the case of disposable packaging for a small amount of content such as a single dose, it is important that the packaging be simple in construction with minimal parts and fast and easy to produce, as well as easily machinable. This is the second problem solved by the invention.

A description of the invention as characterized is provided below.

One aspect of the invention relates to a device for containing, dispensing, and applying to an application surface a content in liquid, gel, cream, or paste form, in the closed/associated state prior to use, able to he placed in the open/separated state for use by an opening/separation operation, comprising, with an axis, a shaped semi-rigid wall, a closing wall, and an applicator having an attachment portion and an application portion that are extensions of one another, such that:
the shaped semi-rigid wall comprises a flat peripheral edge and a recess located on one side of the plane of the edge and surrounded by it, having, as extensions of one another and in communication, a content-receiving portion that is deformable by external pressure, an attachment part with inner projection and a protection portion respectively engaging with the attachment portion and the application portion of the applicator,
the closing wall covers the shaped semi-rigid wall, is rigidly secured to the edge, and seals the recess to provide a content vessel, an attachment cavity for the attachment portion of the applicator, and a protective enclosure for the application portion of the applicator which is adapted, during the opening/separation operation, to separate from said application portion in order to expose it,
a breakable element of the shaped semi-rigid wall and of the closing wall is located transversely between the attachment part and the protection portion, and is adapted to break for the opening/separation operation,
a content-transfer passage extends along the axis between and in communication with the content vessel and the application portion of the applicator, so that, in order to use the device in the open/separated state, content is transferred from the content vessel to the application portion of the applicator when external pressure is exerted on the receiving portion of the shaped semi-rigid wall.

This device is such that the content-transfer passage is made with a structure having a radial spacing that ensures there is no peripheral sealing between the outer peripheral surface of the attachment portion of the applicator and the facing inner peripheral surface of the attachment part of the shaped semi-rigid wall, the structure with radial spacing extending in a communicating and continuous manner along the axis, between the content vessel on the one side and the breakable element and application portion of the applicator on the other; and is such that the content to be applied from the application portion of the applicator is supplied from its outer peripheral shell, while the content is capable of passing through the transfer passage and of being applied as though with a brush.

Thus according to one feature, the attachment portion of the applicator is arranged so that the content cannot pass through it along the axis, in particular said attachment portion is solid or without any through-channel or pores. The invention thus solves the problem posed by known dispensers of the prior art.

In one embodiment, the content-transfer passage is made with a structure having radial spacing between, in addition, the outer peripheral surface of the attachment portion of the applicator and the facing surface of the closing wall.

In one embodiment, the content-transfer passage essentially comprises, and in particular consists of, the structure having radial spacing between the outer peripheral surface of the attachment portion of the applicator and the facing inner peripheral surface of the attachment part of the shaped semi-rigid wall.

In one embodiment, the content-transfer passage essentially comprises, in particular consists of, at least one channel, and in particular a plurality of channels angularly spaced apart, arranged axially, delimited by the outer peripheral surface of the attachment portion of the applicator and the facing inner peripheral surface of the attachment part of the shaped semi-rigid wall, by the structure with radial spacing, extending in a communicating and continuous manner and opening on one side into the content vessel and on the other side near, in particular adjacent to, the outer peripheral shell of the application portion of the applicator located near the attachment portion of the applicator, in particular on the outer surface of the application portion of the applicator.

In one embodiment, the device is with a plurality of channels arranged at least substantially symmetrically with respect to a median plane of the device, orthogonal to the plane of the peripheral edge of the shaped semi-rigid wall.

In one embodiment, the device comprises at least one channel arranged so as to be positioned either the furthest away radially or the closest radially to the closing wall, and in particular comprises at least two channels arranged so that one is positioned the furthest away radially and the other the closest radially to the closing wall.

In one embodiment, the cross-section of a channel has a shape that is flattened in the peripheral direction of the outer peripheral surface of the attachment portion of the applicator and of the facing inner peripheral surface of the attachment part of the shaped semi-rigid wall.

In one embodiment, a channel has an enlarged section near the content vessel and/or near the outer peripheral shell of the application portion of the applicator. In particular, a channel has, near the outer peripheral shell of the application portion of the applicator, a section enlarged in the peripheral direction of the outer peripheral surface of the attachment portion of the applicator and of the facing inner peripheral surface of the attachment part of the shaped semi-rigid wall, so as to represent at least 20%, particularly at least 40%, more particularly at least 60%, and possibly all or substantially all of the peripheral extent of the outer peripheral shell of the application portion of the applicator or of the outer surface of the application portion of the applicator.

According to one embodiment:
the attachment part of the shaped semi-rigid wall has, at least in its middle longitudinal portion, in particular along all or substantially all of its length, an internal cross-section in the general shape of a U, a U with diverging arms, a V, or a semi-circular shape,
the attachment cavity has, at least in its middle longitudinal section, in particular along all or substantially all of its length, an internal cross-section in the general shape of a square, rectangle, trapezium with its large base towards the closing wall, or a sector of a semi-circle.
the shape of the internal cross-section of the attachment part of the shaped semi-rigid wall and the shape of the internal cross-section of the attachment cavity correspond to the shape of the external cross-section of the attachment portion of the applicator, to form the at least one channel of the content-transfer passage, with the structure having radial spacing.

According to one embodiment:

the attachment portion of the applicator has, at least in its middle longitudinal section, in particular along all or substantially all of its length, an external cross-section of a shape that is generally polygonal or curved, in particular elliptical,
the shape of the external cross-section of the attachment portion of the applicator corresponds to the shape of the internal cross-section of the attachment part of the shaped semi-rigid wall and the shape of the internal cross-section of the attachment cavity, to form the at least one channel of the content-transfer passage, with the structure having radial spacing.

In one embodiment, the device combines the structure having radial spacing and a structure having contact or near-contact between the outer peripheral surface of the attachment portion of the applicator and the facing inner peripheral surface of the attachment part of the shaped semi-rigid wall, arranged to leave open or unobstructed the content-transfer passage formed with the structure having radial spacing, and to fulfill a function of fixed or substantially fixed radial positioning of the attachment portion of the applicator within the attachment cavity.

In one embodiment, the structure with contact or near-contact between the outer peripheral surface of the attachment portion of the applicator and the facing inner peripheral surface of the attachment part of the shaped semi-rigid wall is arranged with localized or linear positioning, in particular in the axial direction and/or in the peripheral or surface direction, and is further arranged with discontinuous positioning, in particular distributed positioning, and with a location that is both peripheral and axial.

According to one embodiment:
the attachment part of the shaped semi-rigid wall has, at least in its middle longitudinal section, in particular along all or substantially all of its length, an internal cross-section in the general shape of a U, a U with diverging arms, a V, or a semi-circular shape,
the attachment cavity has, at least in its middle longitudinal section, in particular along all or substantially all of its length, an internal cross-section in the general shape of a square, rectangle, trapezium with its large base towards the closing wall, or a sector of a semi-circle,
the attachment portion of the applicator has, at least in its middle longitudinal section, in particular along all or substantially all of its length, an external cross-section of a shape that is generally polygonal or curved, in particular elliptical,
the shape of the external cross-section of the attachment portion of the applicator corresponds to the shape of the internal cross-section of the attachment part of the shaped semi-rigid wall and the shape of the internal cross-section of the attachment cavity, to form the structure having contact or near-contact, ensuring contact or near-contact between the outer peripheral surface of the attachment portion of the applicator and the facing inner peripheral surface of the attachment part of the shaped semi-rigid wall, the contact or near-contact being arranged so as to provide the structure having radial spacing.

In one embodiment, the attachment part of the shaped semi-rigid wall has an internally projecting structure arranged to leave open or unobstructed the content-transfer passage formed with the structure having radial spacing, and to fulfill a function of fixed or substantially fixed axial positioning of the attachment portion of the applicator within the attachment cavity.

In one embodiment, the internally projecting structure essentially comprises, in particular consists of, at least one projection provided on the inner peripheral surface of the attachment part of the shaped semi-rigid wall, directed towards the axis, and engaging with the outer peripheral surface and/or at least one of the transverse end faces of the attachment portion of the applicator so as to ensure the fixed or substantially fixed axial positioning of the attachment portion of the applicator within the attachment cavity.

In one embodiment, a projection of the internally projecting structure is arranged in a transverse location.

In one embodiment, a projection of the internally projecting structure is located on only a portion of the inner peripheral surface of the attachment part between its two opposing edges. According to another embodiment, a projection of the internally projecting structure is arranged so that content can pass through it in the direction of the axis of the device, it being provided with a through-passage or pores or being formed of discontinuous portions.

In one embodiment, the internally projecting structure comprises two projections, one near the content vessel and the other near the breakable means, engaging with the two transverse end faces of the applicator attachment portion positioned between the two projections.

In one embodiment, the breakable element comprises a frangible line or area of lower resistance, located in a region of the device where the transverse width is smallest within the plane of the peripheral edge of the shaped semi-rigid wall.

In one embodiment, the breakable element is of the snap-and-twist type.

In one embodiment, the applicator comprises an attachment portion such as a rod, generally cylindrical in shape, and an application portion such as a brush, a pad, a spreader, or a wand, in particular an application portion suitable for an application that is precise in size, shape, and limits, and that is small in dimensions, surface area, and amount.

In one embodiment, the shaped semi-rigid wall is a thermoformed wall and the closing wall is a flat coverplate or a second thermoformed wall that is flat or with one or more recesses.

According to one development of the invention, the dosing wall has an integrated or fixedly related secondary or supplemental container for secondary or supplemental content, in particular such as a flexible bag.

According to another development, the shaped semi-rigid well comprises a second recess, separate and spaced apart, closed off by a closing wall, so as to form a secondary or supplemental container for secondary or supplemental content.

According to another development, one or more similar devices are provided so as to form a sheet of devices, in particular side by side, in particular separated from each other by breakable means.

In one embodiment, the content vessel has capacity for a single use, in particular between about 0.5 ml and about 10 ml.

In one aspect, the device is in the state where the content vessel is empty of content. In another aspect, the device is in the state where the content vessel is filled with content such as a cosmetic product, a personal care product, a personal hygiene product, a medication, a cleaning product, a decorating product, a writing product, a masking product, a paint, an ink, a lacquer, or a varnish.

The invention also relates to a device as described, placed in the open/separated state for use, by an opening/separation operation whereby the breakable part is broken, comprising two portions separated from one other:
a usage portion which comprises the content vessel, the attachment cavity tor the attachment portion of the applicator, and the applicator of which the application portion, exposed and protruding from the attachment part, is uncovered and can be applied to the application surface,
a disposable portion which comprises the protective enclosure from which the application portion of the applicator has been extracted and which cannot be placed back on the usage portion.

According to one feature, to make use of the device once it is in the open/separated state, the content is transferred from the content vessel to the application portion of the applicator externally by, firstly, a pressure exerted briefly on the receiving portion of the shaped semi-rigid wall to initiate the transfer, then the movement of the application portion of the applicator on and in contact with the application surface

We will now briefly describe the figures in the drawings.

FIG. 1 is an elevational view from the side of the shaped semi-rigid wall, of a dose of content packaged in one possible embodiment of a device for containing, dispensing, and applying such content on an application surface, in the closed/associated state prior to use, specifically showing that the shaped semi-rigid wall comprises a peripheral edge, a breakable means, and, as extensions of one another, a content-receiving portion, an attachment part with an internally projecting structure comprising two projections, one near the content vessel and the other near the breakable means, and a protection portion.

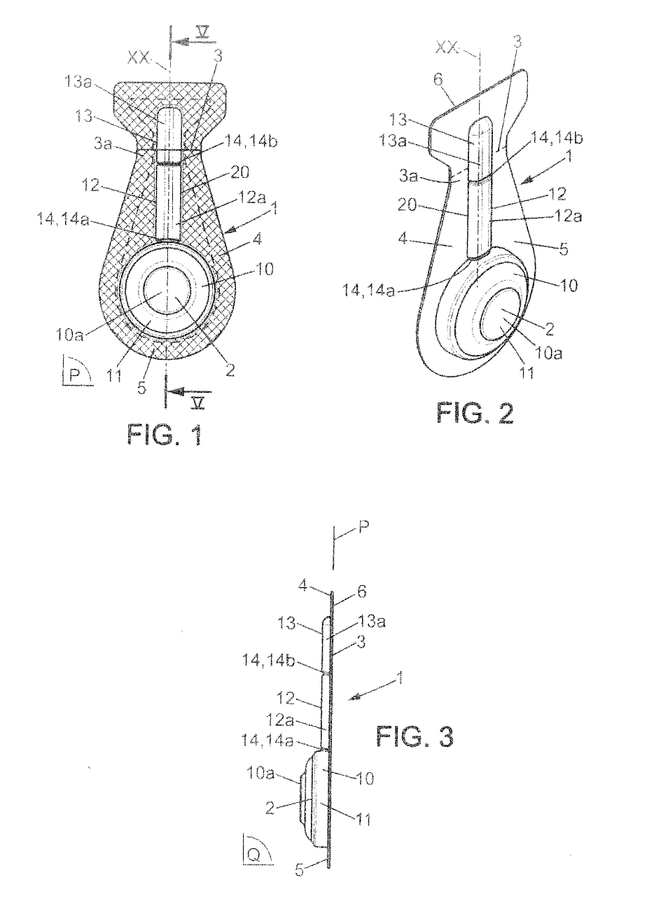

FIG. 2 is a perspective view of the device of FIG. 1, from the side of the shaped semi-rigid wall.

FIG. 3 is a side view of the device of FIG. 1, specifically showing the shaped semi-rigid wall and the closing wall, here a flat coverplate.

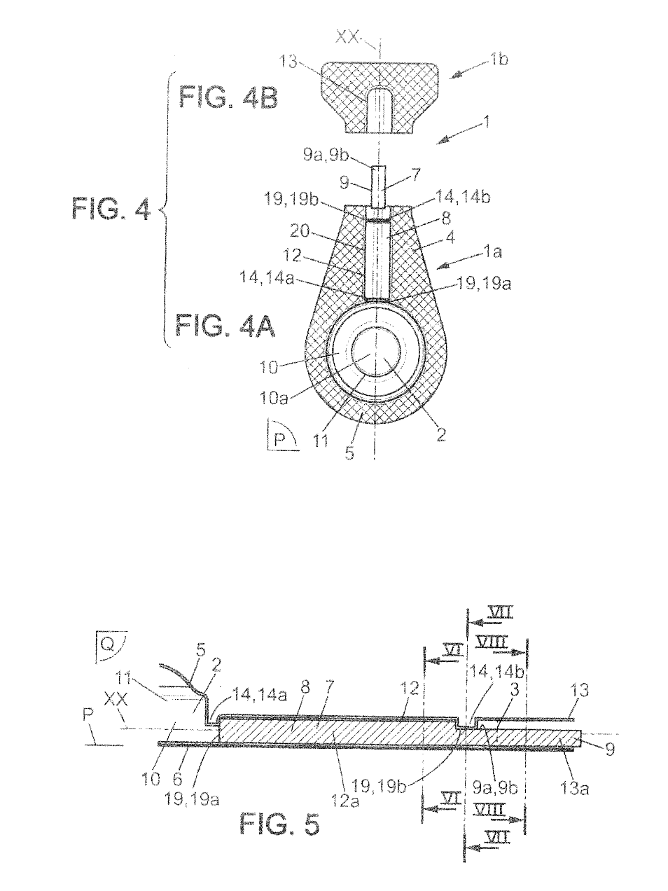

FIG. 4 is an elevational view of the device of FIG. 1 in the open/separated state, containing FIG. 4A showing the usage portion of the device comprising the content vessel, the attachment cavity for the attachment portion of the applicator, and the application portion of the applicator, exposed, projecting from the attachment part, uncovered and able to applied to an application surface, and FIG. 4B showing the disposable portion of the device comprising the protective enclosure from which the application portion of the applicator has been extracted.

FIG. 5 is a partial axial sectional view of the device, along VV of FIG. 1, showing in particular: firstly, the end wall; secondly, for the shaped semi-rigid wall, a portion of the content-receiving portion, the attachment part with an internally projecting structure comprising two projections, one near the content vessel and the other near the breakable means, engaging with the two transverse end faces of the attachment portion of the applicator positioned between the two projections, and the protection portion; thirdly, the attachment portion of the applicator, arranged so that the content cannot pass through it; fourthly, the applicator with its attachment portion in the attachment part and its application portion in the protection portion; and finally the content passage near the outer peripheral shell and more particularly on the outer surface of the application portion of the applicator.

FIG. 6 is a cross-sectional view of the device along line VI-VI of FIG. 5, showing in particular: firstly, that the content-transfer passage is made with a structure having radial spacing that ensures there is no peripheral seal between the outer peripheral surface of the attachment portion of the applicator and the inner peripheral surface of the attachment part of the shaped semi-rigid wall; secondly, that the attachment portion of the applicator is arranged so that the content cannot pass therethrough; thirdly, that the transfer passage here comprises four channels symmetrical about the median plane of the device, in other words with two channels radially apart from and two channels radially adjacent to the closing wall, these channels having a cross-sectional shape that is flattened in the peripheral direction of the outer peripheral surface of the attachment portion and of the inner peripheral surface of the attachment part; and fourthly that the structure having radial spacing is combined with a structure having contact or near-contact between the outer peripheral surface of the attachment portion and the inner peripheral surface of the attachment part.

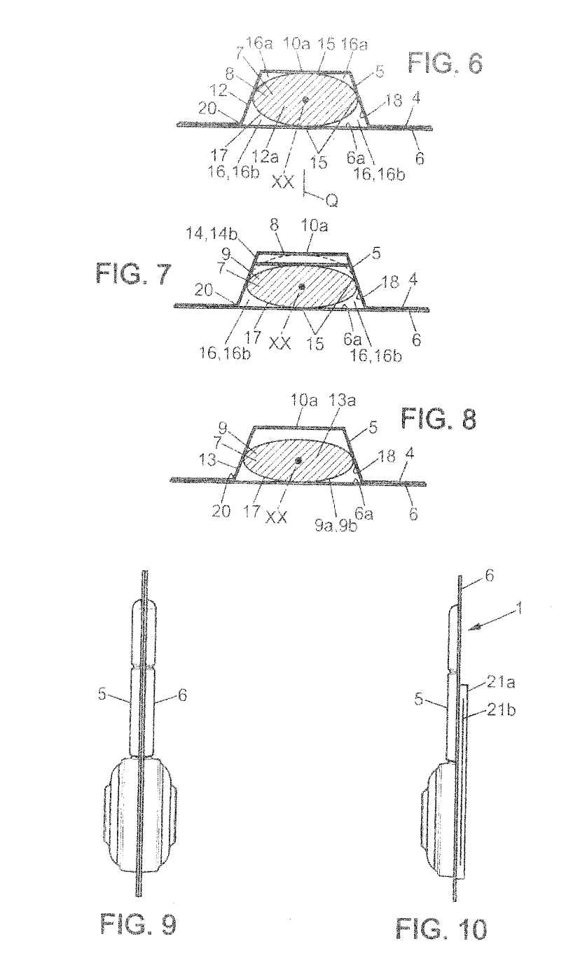

FIG. 7 is a cross-sectional view similar to FIG. 6. along line VII-VII of FIG. 5, showing in particular the projection of the internally projecting structure located near the breaking means, engaging with the corresponding transverse end face of the attachment portion of the applicator.

FIG. 8 is a cross-sectional view similar to FIG. 8. along line VIII-VIII of FIG. 5, showing in particular that the content passage distributes the content toward the outer peripheral shell and more particularly on the outer surface of the application portion of the applicator.

FIG. 9 is a side view of the device, here with a closing wall that is a second thermoformed wall with recesses, symmetrical to the first.

FIG. 10 is a side view of the device of FIG. 1, in the case where the closing wall has an integrated or fixedly related secondary or supplemental container for secondary or supplemental content, such as a flat bag.

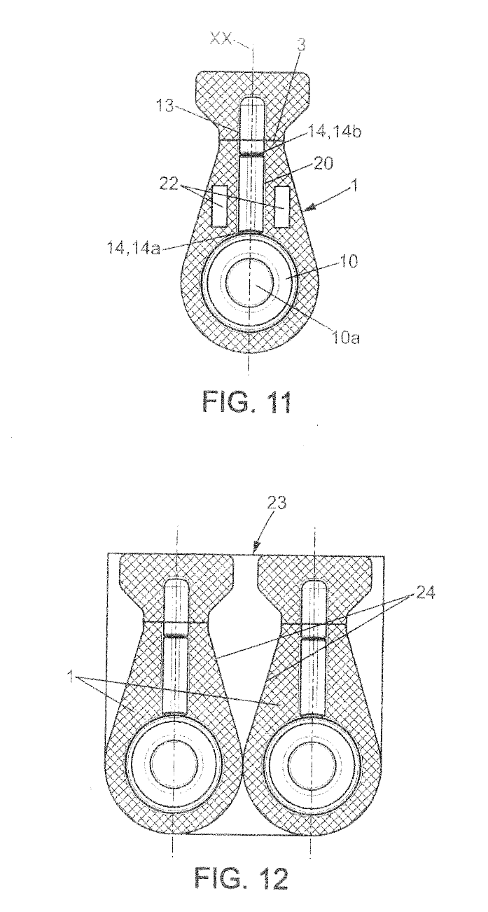

FIG. 11 is an elevational view from the side of the shaped semi-rigid wall of the device, where the shaped semi-rigid wall comprises two second walls with recesses, separate and spaced apart, closed off, so as to form secondary or supplemental containers.

FIG. 12 is an elevational view from the side of the shaped semi-rigid wall, showing a device arranged with another similar device so as to form a sheet of side-by-side devices separated by breakable means.

Below is a detailed description, with examples and references to the drawings, of several embodiments of a device 1 for containing, dispensing, and applying to an application surface, in particular applying as though with a brush, a content in liquid, gel, cream, or paste form.

In the context of the invention, the device 1 is specifically adapted to receive a dose of the content in an amount that may be appropriate for a single use, in particular an amount of between 0.5 ml and 10 ml.

This content is, for example, a cosmetic product, a personal care product, a personal hygiene product, a medication, a cleaning product, a decorative product, a writing product, a masking product, a paint, an ink, a lacquer, or a varnish. This list is not exhaustive.

The application surface may be skin, nails, lips, a product to be maintained, to be decorated, on which one wishes to write, which one wants to mask, to which one wants to apply paint, lacquer, or varnish. This list is not exhaustive.

The invention relates to both the situation where the content vessel 2 of the device 1 is empty of content and the situation where the vessel 2 is filled with content.

Before use, the device 1 is in the closed/associated state (see FIGS. 1-3, 5-8 in particular). It can be placed in the open/separated state (see FIG. 4) for use, by means of an opening/separation operation, the device 1 comprising breaking means 3 in the form of a frangible line or area of lower resistance, especially of the type known as snap and twist.

The device 1 is such that, in the open/separated state. It comprises a usage portion 1a and a disposable portion 1b which cannot be placed back on the usage portion 1a.

In the context of the invention, the device 1 is specifically adapted for content application that is precise in terms of size, shape, and limits, and small in terms of dimensions, surface area, amount.

In addition, the device 1 is of a type that can portable, compact, lightweight, and small—for example a few centimeters for the largest dimension—which can be stored in a pocket, bag, trinket dish, or drawer.

The device 1 has an axis XX and has an extension plane P which is the plane of a flat peripheral edge 4 of a shaped semi-rigid wall 5. More particularly, the extension plane P is parallel to the plane of FIGS. 1 and 4. The device 1 has a general median plane Q of symmetry that is orthogonal to plane P and passes through axis XX. More particularly, the median plane Q is parallel to the plane of FIGS. 3 and 5.

The term “axial”, the term “radial”, and the expression “angularly spaced apart” are understood to have their conventional meaning, here in reference to axis XX. The term “transverse” refers to that which is perpendicular or orthogonal to axis XX. The term “peripheral” refers to that which is generally located on an external perimeter surrounding axis XX.

The device 1 comprises three parts, namely the shaped semi-rigid wall 5, here a thermoformed wall, the closing wall 6, and the applicator 7. The applicator 7 is formed of an attachment portion 8, such as a rod, which may be generally cylindrical, and an application portion 9 which is an axial extension of the attachment portion, such as a brush, a pad, a scoop, or a wand, and in particular an application portion suitable for an application that is precise in size, shape, and limits, and small in dimensions, surface area, and amount. The applicator 7 is a single part or multiple assembled parts, in particular two, for example the attachment portion 8 and the application portion 9.

The shaped semi-rigid wall 5 comprises the peripheral edge 4 and a recess 10.

The recess 10 is located on one side of plane P and Is surrounded by the edge 4.

The recess 10 has, as extensions of one another and in communication with one another, a receiving portion 11, an attachment part 12, and a protection portion 13.

The closing wall 6 covers the shaped semi-rigid wall 5, is rigidly attached to the edge 4, and seals the recess 10 closed.

In the embodiment of FIGS. 1 to 8 and 10 to 12, the closing wall 6 is a tight coverplate, flat or substantially flat. In the embodiment of FIG. 9, the closing wall 6 is a second thermoformed wall that is flat or shaped with one or more recesses, particularly similar to the shaped semi-rigid wall 5, more particularly identical to said wall, and arranged symmetrically to extension plane P.

With the receiving portion 11 of the recess 10, the closing wall 6 defines a content vessel 2. With the attachment part 12 of the recess 10, it defines an attachment cavity 12a. With the protection portion 13 of the recess 10, it defines a protective enclosure 13a.

The receiving portion 11, respectively the content vessel 2, here with a shape close to that of a spherical sector, is adapted to receive the dose of content.

The receiving portion 11 is deformable by means of external pressure orthogonal to plane P, such as pressure exerted by a user's fingers.

The attachment part 12, elongated and rectilinear, is provided with an internally projecting structure 14 which acts to fix or substantially fix the axial positioning of the attachment portion 8 of the applicator 7 within the attachment cavity 12a, and therefore more generally of the applicator 7 relative to the shaped semi-rigid wall 5.

The attachment part 12 is part of a structure having contact or near-contact 15 which acts to fix or substantially fix the radial positioning of the attachment portion 8 of the applicator 7 within the attachment cavity 12a, and therefore more generally of the applicator 7 relative to the shaped semi-rigid wall 5.

The attachment part 12, respectively the attachment cavity 12a, and the protection portion 13, respectively the protective enclosure 13a, respectively engage with the attachment portion 8 and the application portion 9 of the applicator 7. The protective enclosure 13a is adapted, during the opening/separation operation, to separate from the application portion 9 of the applicator 7 in order to expose it, as shown in FIG. 4.

The breakable element 3 concerns the shaped semi-rigid wall 5 and the closing wall 6. The breakable element 3 is located transversely between the attachment portion 12 and the protection portion 13, in particular is located in a region 3a of the device 1 where the transverse width is smallest within the extension plane P.

The device 1 also comprises a content-transfer passage 16 made with a structure having radial spacing. This passage 16 is permanent and is created during manufacture.

The passage 16 extends along axis XX between and in communication with the content vessel 2 and the application portion 9 of the applicator 7.

To use the device 1 in the open/separated state, content is transferred from the content vessel 2 to the application portion 9 of the applicator 7, externally to the applicator, after external pressure is exerted on the receiving portion 11 of the shaped semi-rigid wall 5.

The structure with radial spacing ensures there is no peripheral sealing between the outer peripheral surface 17 of the attachment portion 8 of the applicator 7 and the facing inner peripheral surface 18 of the attachment part 12 of the shaped semi-rigid wall 5.

In one embodiment, the passage 16 essentially comprises, in particular consists of, the structure having radial spacing between the outer peripheral surface 17 and the inner peripheral surface 18.

According to one possibility, the passage 16 is formed with a structure having further radial spacing between the outer peripheral surface 17 of the attachment portion 8 of the applicator 7 and the facing inner surface 6a of the closing wall 6 forming a coverplate.

The structure having radial spacing extends in a communicating and continuous manner along axis XX, between the content vessel 2 on the one side, and the breaking means 13 and application portion 9 of the applicator 7 on the other.

The structure is thus arranged so that the content to be applied from the application portion 8 of the applicator 7 is supplied from its outer peripheral shell 9a, which is generally cylindrical for example.

The content is able to pass through the transfer passage 16.

Conversely, the device 1 is such that the attachment portion 8 of the applicator 7 is arranged so that content cannot pass it along axis XX, in particular because the attachment portion 3 is solid or has no through-channel or pores.

Thus, as represented more particularly in FIGS. 6 and 7, the passage 16 comprises four channels, namely two channels 16a and two channels 16b angularly spaced apart and arranged axially, delimited by the outer peripheral surface 17 of the attachment portion 8 of the applicator 7 and the facing inner peripheral surface 18 of the attachment part 12 of the shaped semi-rigid wall 5, by means of the structure having radial spacing. These channels 16a, 16b extend in a communicating and continuous manner and on one end open into the content vessel 2, and on the other side end near and in particular adjacent to the outer peripheral shell 9a of the application portion 7 of the applicator 9, located near the attachment portion 8 of the applicator 7, and even more particularly on the outer peripheral surface 9b of the application portion 8 of the applicator 8.

The two channels 16a and the two channels 16b are respectively arranges symmetrically to the median plane Q. Two channels 16a are arranged to be the furthest apart radially from the closing wall 6, being positioned near the bottom 10a of the recess 10 opposite the edge 4.

Two channels 16b are arranged to be the closest radially to the closing wall 6, being positioned near the edge 4.

This embodiment does not exclude others in which the passage 16 essentially comprises, in particular consists of, at least one channel and m particular a plurality of channels angularly spaced apart.

As is more particularly represented in FIGS. 6 and 7, the cross-section of a channel 16a, 16b has a flattened shape extending in the peripheral direction of the outer peripheral surface 17 of the attachment portion 9 of the applicator 7 and of the facing inner peripheral surface 18 of the attachment part 12 of the shaped semi-rigid wall 5.

According to one possibility, a channel 16a, 16b is provided having an enlarged section near the content vessel 2, to facilitate entry of the content into the channel 16a, 16b from the content vessel 2.

Additionally or alternatively, a channel 16a, 16b is provided having an enlarged section near the outer peripheral shell 9a of the application portion 9 of the applicator 7, to facilitate the distribution of content on the application portion 9. In particular, this enlarged section represents at least 20%, particularly at least 40%, more particularly at least 60%, and possibly all or substantially all of the peripheral extent of the outer peripheral shell 9a of the application portion of the applicator or of the outer surface 9b of the application portion 9 of the applicator 7.

The internally projecting structure 14 and the structure with contact or near-contact 15 are arranged to leave open or unobstructed the passage 16, here the channels 16a, 16b.

The structure with contact or near-contact 15, aside from acting to fix or substantially fix the radial positioning of the attachment portion 8 of the applicator 7 within the attachment cavity 12a, contributes to forming the structure having radial spacing and to the creation of the passage 16 and the channels 16a and 16b.

In the embodiment shown in FIG. 6, firstly the attachment part 12 of the shaped semi-rigid wall 5 has an internal cross-section in the general shape of a U with diverging arms extending towards the opening opposite the bottom 10a, the attachment cavity 12a thus having an internal cross-section that is generally trapezoidal with its large base towards the closing wall 6 and its small base towards the bottom 10a.

Secondly, the attachment portion 8 of the applicator 7 has an external cross-section that is generally ellipsoidal in shape.

Thirdly, the shape of the internal cross-section of the attachment part 12 of the shaped semi-rigid wall 5 and the shape of the internal cross-section of the attachment cavity 12a, on the one hand, and the shape of the external cross-section of the attachment portion 8 of the applicator 7, on the other hand, correspond to form the channels 16a and 16b, with the structure having radial spacing, and to form the structure having contact or near-contact 15 which is arranged to define the structure having radial spacing, as explained above.

This embodiment is not exclusive of others, as long as the functions described above are provided.

Thus, more generally, the attachment part 12 of the shaped semi-rigid wall 5 has, at least in its middle longitudinal section, in particular along all or substantially all of its length, an internal cross-section where the general shape may also be a U, a V, or a semi-circular shape. And the attachment cavity 12a has, at least in its middle longitudinal section, in particular along all or substantially all of its length, an internal cross-section where the general shape may also he a square, rectangle, or a sector of a semi-circle. And the attachment portion 8 of the applicator 7 has, at least in its middle longitudinal section, in particular along all or substantially all of its length, an external cross-section which may also be of a shape that is generally polygonal or curved.

In the embodiment represented in FIG. 6, the structure with contact or near-contact 15 is arranged into a quadrupling substantially linear arrangement (or having little contact surface area in the peripheral direction) in the axial direction, which leads to an arrangement divided into four areas.

This embodiment is not exclusive of others in which the structure with contact or near-contact 15 is arranged in a localized arrangement in the axial direction and/or in the peripheral direction, or on the surface, or is further arranged in a discontinuous arrangement.

Generally, the internally projecting structure 14 essentially comprises, in particular consists of, at least one inner projection arranged with a transverse position, provided on the inner peripheral surface 18 of the attachment part 12 of the shaped semi-rigid wall 5, directed towards axis XX, and engaging with the outer peripheral surface 17 and/or at least one of the transverse end faces 19 of the attachment portion 8 of the applicator 7.

In general and depending on the embodiment, a projection of the internally projecting structure 14 is located on only a portion of the inner peripheral surface 18 of the attachment part 12 between its two opposing edges 20, or is arranged so that content can pass through it in the direction of axis XX, it being provided with a through-passage or pores or being formed of discontinuous portions.

In the particular embodiment specifically represented in FIGS. 1 to 5 and 8, the infernally projecting structure 14 comprises two projections, one 14a near the content vessel 2 and the other 14b near the breakable element 3. These two projections 14a and 14b match the respective two transverse end faces 19a, 19b of the attachment portion 8 of the applicator 7, said attachment portion 8 being positioned between the two projections 14a and 14b so that it is axially immobilized.

According to one development shown in FIG. 10, the closing wall 6 is integrated with or fixedly related to one (or more) secondary or supplemental containers 21a for one (or more) secondary or supplemental contents 21b. Such a secondary or supplemental container 21a may be, in particular, a flexible bag glued or welded to the outer face of the closing wall 6.

According to another development shown in FIG. 11, the shaped semi-rigid wall 5 comprises one (or more) second recesses 22, separate and spaced apart from recess 10, closed off by a closing wall which may be closing wall 6, so as to form one (or more) secondary or supplemental containers for one (or more) secondary or supplemental contents.

According to another development shown in FIG. 12, one (or more) other devices 1 are provided, similar to the device 1 previously described, so as to form a sheet 23 of devices, in particular side by side, in particular separated from each other by breakable element 24.

A device 1 as described may be placed in the open/separated state for use, by means of an opening/separation operation whereby the breakable portion 3 is broken (FIG. 4). It then comprises two portions separated from one another, namely the usage portion 1a and the disposable portion 1b.

According to one feature, to make use of the device 1 once it is in the open/separated state, the content is transferred from the content vessel 2 to the application portion 9 of the applicator 7 by means of, firstly, pressure exerted briefly on the receiving portion 11 of the shaped semi-rigid wall 5 to initiate the transfer, then by the movement of the application portion 9 of the applicator 7 on and in contact with the application surface.

1. The device, for containing, dispensing, and applying to an application surface a content in liquid, gel, cream, or paste form, in the closed/associated state prior to use, able to be placed in the open/separated state for use by an opening/separation operation, comprising, with an axis, a shaped semi-rigid wall, a closing wall, and an applicator having an attachment portion and an application portion that are extensions of one another, such that:
the shaped semi-rigid wall comprises a flat peripheral edge and a recess located on one side of the plane of the edge and surrounded by it, having, as extensions of one another and in communication, a content receiving portion that is deformable by external pressure, an attachment part with inner projection and a protection portion respectively engaging with the attachment portion and the application portion of the applicator,
the recess to provide a content vessel, an attachment cavity for the attachment portion of the applicator, and a protective enclosure for the application portion of the applicator which is adapted, during the opening/separation operation, to separate from said application portion in order to expose it.
a breakable element of the shaped semi-rigid wall and of the closing wall is located transversely between the attachment part and the protection portion, and is adapted to break for the opening/separation operation,
a content-transfer passage extends along the axis between, and in communication with, the content vessel and the application portion of the applicator, so that, in order to use the device in the open/separated state, content is transferred from the content vessel to the application portion of the applicator when external pressure is exerted on the receiving portion of the shaped semi-rigid wall,
characterized in that the content-transfer passage is made with a structure having radial spacing that ensures there is no peripheral sealing between the outer peripheral surface of the attachment portion of the applicator and the facing inner peripheral surface of the attachment part of the shaped semi-rigid wall, the structure with radial spacing extending in a communicating and continuous manner along the axis, between the content vessel on the one side, and the breakable element and application portion of the applicator on the other, and in that the content to be applied from the application portion of the applicator is supplied from its outer peripheral shell, while the content is capable of passing through the transfer passage and of being applied as though with a brush.

2. The device according to claim 1, wherein the attachment portion of the applicator is arranged so that the content cannot pass through it along the axis, in particular wherein said attachment portion is solid without any through-channel or pores.

3. The device according to claim 1, wherein the content-transfer passage is made with a structure having radial spacing between the outer peripheral surface of the attachment portion of the applicator and the facing inner surface of the closing wall.

4. The device according to claim 1, wherein the content-transfer passage essentially comprises, in particular consists of, at least one channel, and in particular a plurality of channels angularly spaced apart, arranged axially, delimited by the outer peripheral surface of the attachment portion of the applicator and the facing inner peripheral surface of the attachment part of the shaped semi-rigid wall, by the structure with radial spacing, extending in a communicating and continuous manner and opening on one side into the content vessel and on the other side near, in particular adjacent to, the outer peripheral shell of the application portion of the applicator located near the attachment portion of the applicator, in particular on the outer surface of the application portion of the applicator.

5. The device according to claim 4, comprising a plurality of channels arranged at least substantially symmetrically with respect to a median plane of the device, orthogonal to the plane of the peripheral edge of the shaped semi-rigid wall, and/or comprising at least one channel arranged so as to be positioned either the furthest away radially or the closest radially to the closing wall, and in particular comprising at least two channels arranged so that one is positioned the furthest away radially and the other the closest radially to the closing wall.

6. The device according claim 4, wherein the cross-section of a channel has a shape that is flattened in the peripheral direction of the outer peripheral surface of the attachment portion of the applicator and of the facing inner peripheral surface of the attachment part of the shaped semi-rigid wall.

7. The device according to claim 4, wherein a channel has, near the outer peripheral shell of the application portion of the applicator, a section enlarged in the peripheral direction of the outer peripheral surface of the attachment portion of the applicator and of the facing inner peripheral surface of the attachment part of the shaped semi-rigid wall, so as to represent at least 20%, in particular at least 40%, more particularly at least 60%, and possibly all or substantially all of the peripheral extent of the outer peripheral shell of the application portion of the applicator or of the outer surface of the application portion of the applicator.

8. The device according to claim 4, wherein:
the attachment part of the shaped semi-rigid wall has, at least in its middle longitudinal section, in particular along all or substantially all of its length, an internal cross-section in the general shape of a U, a U with diverging arms, a V, or a semi-circular shape,
the attachment cavity has, at least in its middle longitudinal section, in particular along all or substantially all of its length, an internal cross-section in the general shape of a square, rectangle, trapezium with its large base towards the closing wall, or a sector of a semi-circle,
the shape of the internal cross-section of the attachment part of the shaped semi-rigid wall and the shape of the internal cross-section of the attachment cavity correspond to the shape of the external cross-section of the attachment portion the applicator, to form the at least one channel of the content-transfer passage, with the structure having radial spacing.

9. The device according to claim 4, wherein:
the attachment portion of the applicator has, at least in its middle longitudinal section, in particular along all or substantially all of its length, an external cross-section of a shape that is generally polygonal or curved, in particular elliptical,
the shape of the external cross-section of the attachment portion of the applicator corresponds to the shape of the internal cross-section of the attachment, part of the shaped semi-rigid wall and the shape of the internal cross-section of the attachment cavity, to form the at least one channel of the content-transfer passage, with the structure having radial spacing.

10. The device according to claim 1, which combines the structure having radial spacing and a structure having contact or near-contact between the outer peripheral surface of the attachment, portion of the applicator and the facing inner peripheral surface of the attachment part of the shaped semi-rigid wall, arranged to leave open or unobstructed the content-transfer passage formed with the structure having radial spacing, and to fulfill a function of fixed or substantially fixed radial positioning of the attachment portion of the applicator within the attachment cavity.

11. The device according to claim I, wherein:
the attachment part of the shaped semi-rigid wall has, at least in its middle longitudinal section, in particular along all or substantially all of its length, an internal cross-section in the general shape of a U, a U with diverging arms, a V, or a semi-circular shape,
the attachment cavity has, at least in its middle longitudinal section, in particular along all or substantially ail of its length, an internal cross-section in the general shape of a square, rectangle, trapezium with its large base towards the closing wall, or a sector of a semi-circle,
the attachment portion of the applicator has, at least in its middle longitudinal section, in particular along all or substantially all of its length, an external cross-section of a shape that is generally polygonal or curved, in particular elliptical,
the shape of the external cross-section of the attachment portion of the applicator corresponds to the shape of the internal cross-section of the attachment part of the shaped semi-rigid wall and the shape of the internal cross-section of the attachment cavity, to form the structure having contact or near-contact, ensuring contact or near-contact between the outer peripheral surface of the attachment portion of the applicator and the facing inner peripheral surface of the attachment part of the shaped semi-rigid wall, the contact or near-contact being arranged so as to provide the structure having radial spacing.

12. The device according to claim 1, wherein the attachment part of the shaped semi-rigid wall has an internally projecting structure arranged to leave open or unobstructed the content-transfer passage formed with the structure having radial spacing, and to fulfill a function of fixed or substantially fixed axial positioning of the attachment portion of the applicator within the attachment cavity.

13. The device according to claim 12, wherein the internally projecting structure essentially comprises, in particular consists of at least one projection provided on the inner peripheral surface of the attachment part of the shaped semi-rigid wall, directed towards the axis, arranged with a transverse location and engaging with the outer peripheral surface and/or at least one of the transverse end faces of the attachment portion of the applicator so as to ensure the fixed or substantially fixed axial positioning of the attachment portion of the applicator within the attachment cavity.

14. The device according to claim 12, wherein a projection of the internally projecting structure is located on only a portion of the inner peripheral surface of the attachment part between its two opposing edges or is arranged so that content can pass through it in the direction of the axis of the device, it being provided with a through-passage or pores or being formed of discontinuous portions.

15. The device according to claim 12, wherein the internally projecting structure comprises two projections, one near the content vessel and the other near the breakable means, engaging with the two transverse end faces of the applicator attachment portion positioned between the two projections.

16. The device according to claim 1, wherein the breakable element is of the snap and twist type.

17. The device according to claim 1, wherein the applicator comprises an attachment portion such as a rod, generally cylindrical in shape, and an application portion such as a brush, a pad, a spreader, or a wand, in particular an application portion suitable for an application that is precise in size, shape, and limits,, and that is small in dimensions, surface area, and amount.

18. The device according to claim 1, wherein the shaped semi-rigid wall is a thermoformed wall and wherein, the closing wall is a flat coverplate or a second thermoformed wall that is flat or with one or more recesses.

19. The device according to claim 1, wherein the closing wall has an integrated or fixedly related secondary or supplemental container for secondary or supplemental content, in particular such as a flexible bag.

20. The device according to claim 1, wherein the shaped semi-rigid wall comprises a second recess, separate and spaced apart, closed off by a closing wall, so as to form an secondary or supplemental container for secondary or supplemental content.

21. The device according to claim 1, arranged with one or more other similar devices so as to form a sheet of devices, in particular side by side, in particular separated from each other by breakable means.

22. The device according to claim 1, placed in the open/separated state for use, by an opening/separation operation whereby the breakable portion is broken, comprising two portions separated from one another:
a usage portion which comprises the content vessel, the attachment cavity for the attachment portion of the applicator, and the applicator of which the application portion, exposed and protruding from the attachment part, is uncovered and can be applied to the application surface,
a disposable portion which comprises the protective enclosure from which the application portion of the applicator has been extracted and which cannot be placed back on the usage portion.

23. The device according to claim 1, wherein to make use of the device once it is in the open/separated state, the content is transferred from the content vessel
to the application portion of the applicator by, firstly, a pressure exerted briefly on the receiving portion of the shaped semi-rigid wall to initiate the transfer, then the movement of
the application portion of the applicator on and in contact with the application surface.
